(19) United States
(12) Patent Application Publication
(10) Pub. No.: US 2026/0077159 A1
(43) Pub. Date: Mar. 19, 2026

(54) External Female Catheter

(71) Applicant: HR Pharmaceuticals, Inc., York, PA (US)

(72) Inventor: Matthew Hanna, Lancaster, PA (US)

(73) Assignee: HR Pharmaceuticals, Inc., York, PA (US)

(21) Appl. No.: 18/890,350

(22) Filed: Sep. 19, 2024

(51) Int. Cl. A61M 25/00; A61M 25/01

(52) U.S. Cl. CPC A61M 25/0053; A61M 25/0147; A61M 25/0152; A61M 2025/0161

(57) ABSTRACT

A catheter assembly includes a container vessel with a receiving passageway extending through an upper region of the container vessel. The collection vessel further includes a high absorption member positioned within an inner surface of the container vessel. The collection vessel further includes tube fitted within the receiving passageway.

### FIELD OF THE INVENTION

The present invention pertains to an external catheter and, more particularly, to an improved external catheter for female genitalia.

### BACKGROUND

Catheterization is known to cause problems with nosocomial infections, and generally there are a high number of catheter-related urinary tract infections (CAUTI) annually. CAUTI is one of the most common nosocomial infections in hospitals and nursing homes, accounting for a major percentage of institutionally acquired infections. Catheterization also is an invasive procedure, albeit minimally. Therefore, there is a need to provide a solution for the problems presented above.

### SUMMARY

A catheter assembly includes a container vessel with an overall curvature having an outer rim, the container vessel further includes a receiving passageway extending through an upper region of the container vessel. A collection vessel includes a high absorption member positioned within an inner surface of the container vessel and the collection vessel further includes a tube fitted within the receiving passageway.

### BRIEF DESCRIPTION OF DRAWINGS

In the following, the present invention is described in more detail with references to the drawings in which:

FIG. 1 illustrates a front, top, right side perspective view of the invention;

FIG. 2 illustrates a front, bottom, left side perspective view of the invention of FIG. 1;

FIG. 3 illustrates a rear, bottom, right side perspective view thereof;

FIG. 4 illustrates a rear, top, right side perspective view thereof;

FIG. 5 illustrates a cross sectional view thereof;

FIG. 6 illustrates a front perspective view thereof;

FIG. 7 illustrates a bottom perspective view thereof;

FIG. 8 illustrates a top perspective view thereof;

FIG. 9 illustrates a left side perspective view thereof;

FIG. 10 illustrates a rear perspective view thereof;

FIG. 11 illustrates a cross-sectional view thereof;

FIG. 12 illustrates another cross-sectional view thereof;

FIG. 13 illustrates another cross-sectional view thereof;

FIG. 14 illustrates another cross-sectional view thereof;

FIG. 15 illustrates another cross-sectional view thereof;

FIG. 16 illustrates a rear, top, right side perspective view thereof;

FIG. 17 illustrates an enlarged perspective view thereof; and

FIG. 18 illustrates a cross-sectional view of FIG. 17.

### DETAILED DESCRIPTION OF THE EMBODIMENTS

With reference to the Figures, a catheter assembly 1 according to the invention is shown.

In an exemplary embodiment shown, a catheter assembly 1 according to the invention generally includes a container vessel 10 and a collection vessel 50.

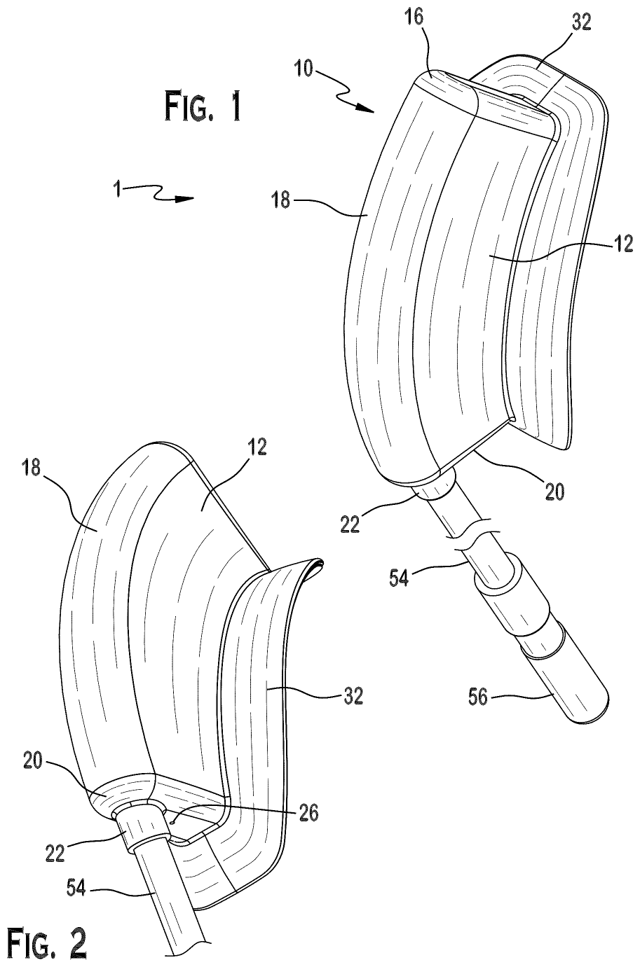

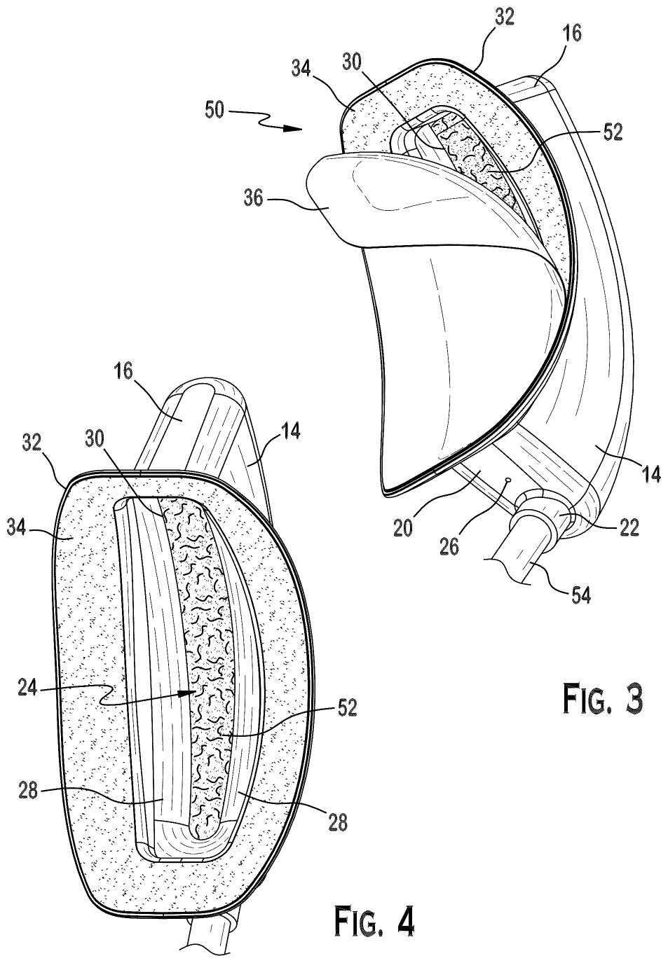

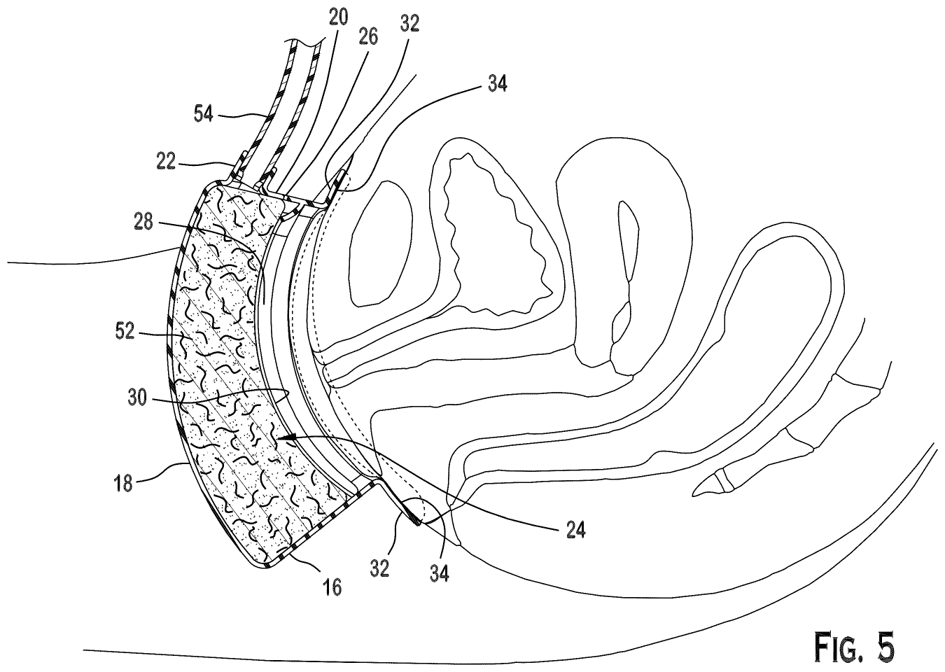

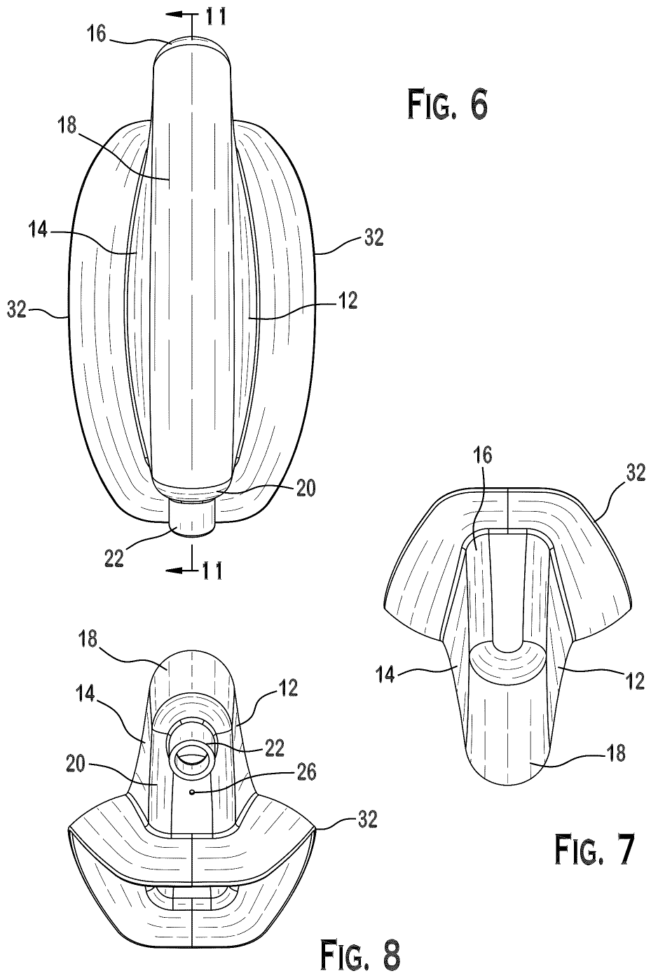

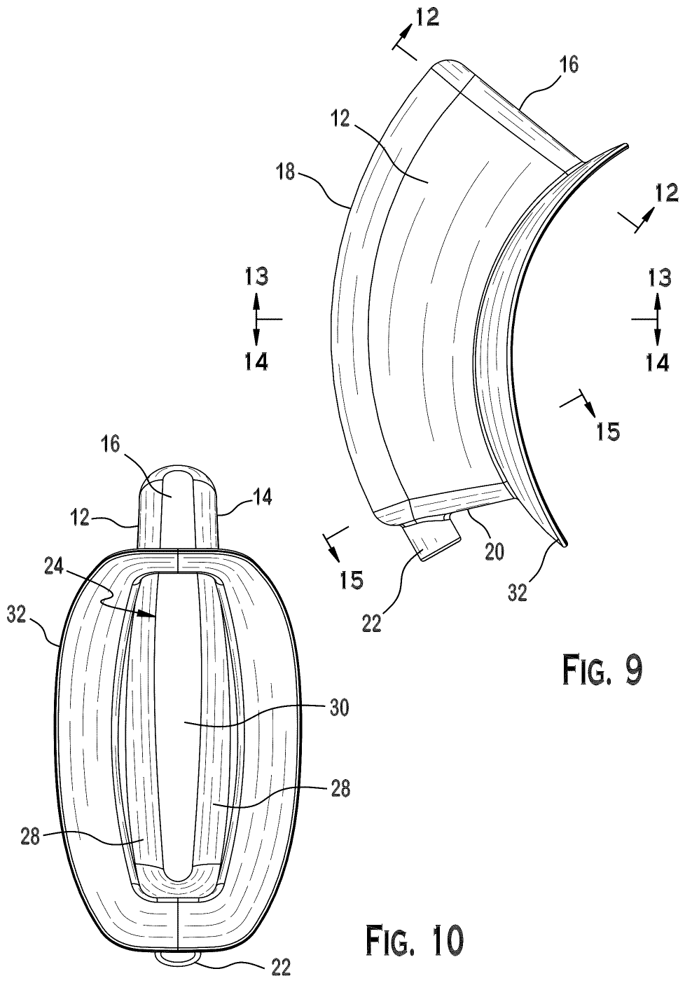

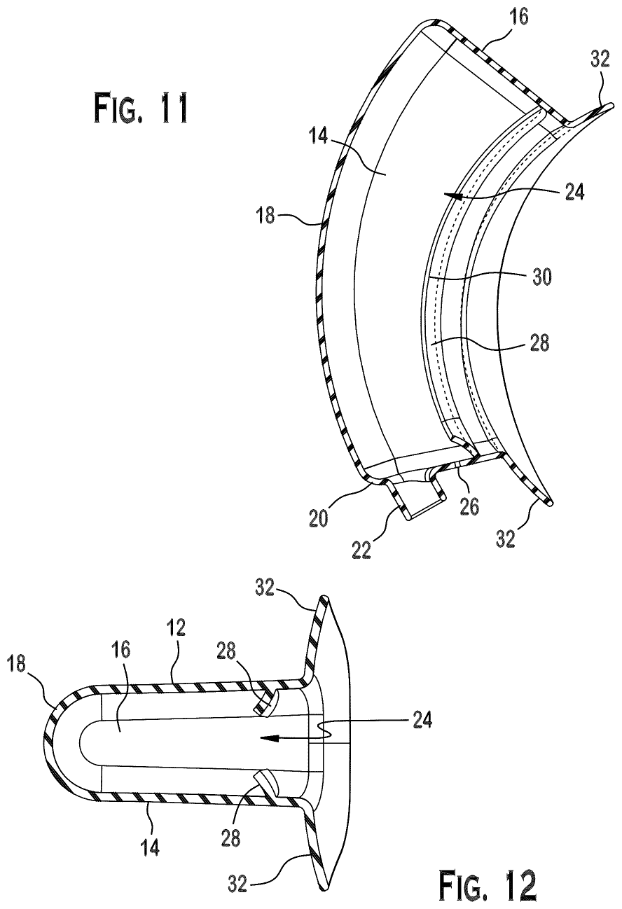

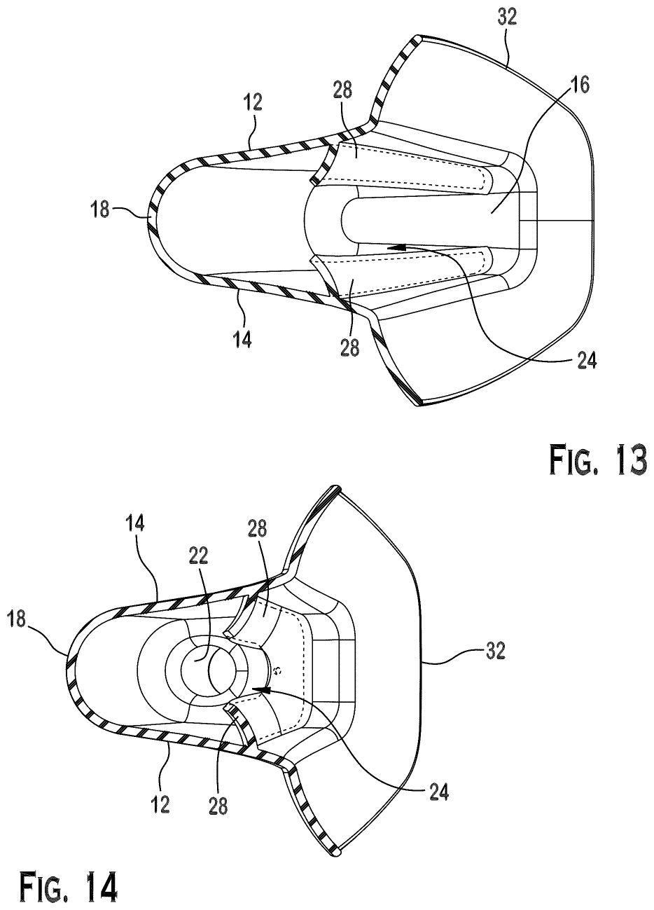

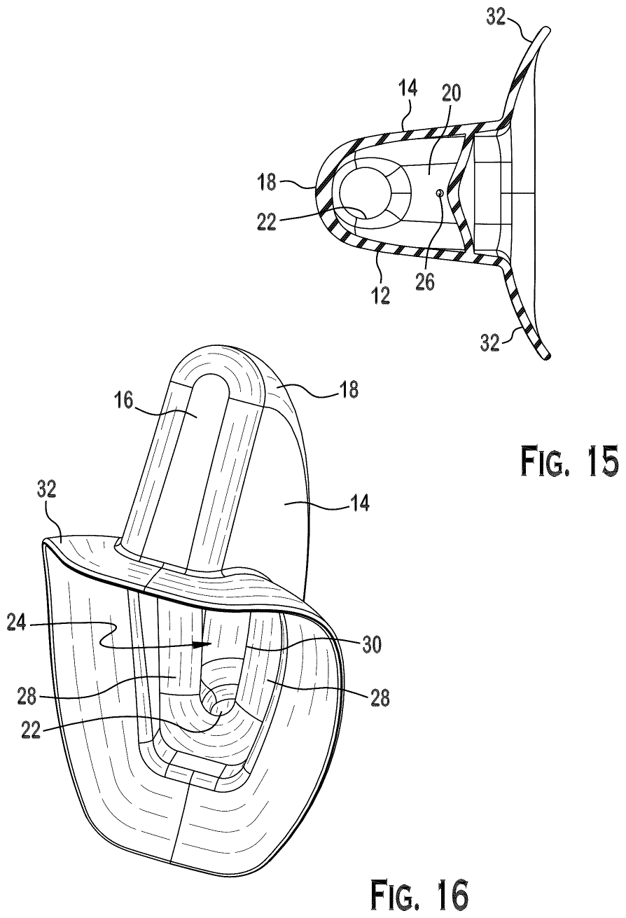

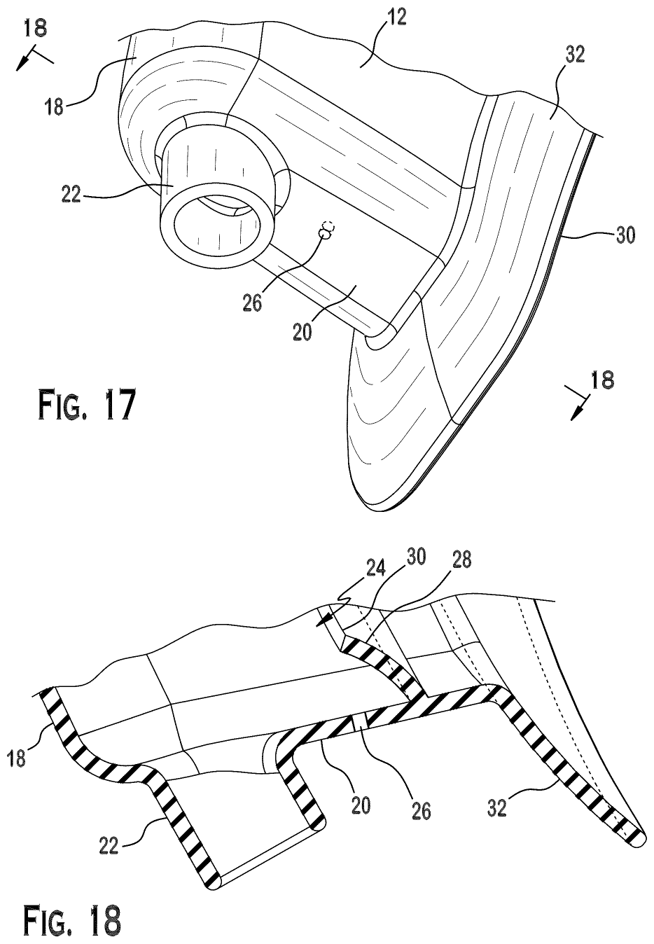

With reference to FIGS. 1-18, an exemplary embodiment of the container vessel 10 is shown and generally includes a first side 12, a second side 14, a third side 16, a fourth side 18 and a fifth side 20. The first side 12 and the second side 14 are elongated bends. The third side 16, fixes the first side 12 and the second side 14 together. The third side 16 further includes a concaved surface along the central portion of the third side 16. The fourth side 18, fixes the first side 12 and the second side 14 together. The fifth side 20, couples the first side 12 and the second side 14 together. The fifth side 20 further includes a release passageway 22 extending through the fifth side 20 and into a receiving channel 24. The release passageway 22 is a cylindrical funnel extending away from the fifth side 20 and positioned at an upper region of the container vessel 10. One skilled in the art would understand the shape of the release passageway is not the exclusive embodiment.

The receiving channel 24 is positioned within the opening of the inner surface of the container vessel 10. The fifth side 20 further includes a recess 26 positioned between the release passageway 22 and an outer rim 32. One skilled in the art would understand the applicant's design is not the exclusive embodiment. The recess 26 is a pressure regulation valve. It allows the pressure to be neutral within the container vessel 10 whether there is fluid present or not within the container vessel 10.

In the exemplary embodiment, the recess 26 is cylindrical in other embodiments it can be a different polygonal shape. One skilled in the art would understand the applicant's design is not the exclusive embodiment.

In the exemplary embodiment, the plurality of sides 12, 14, 16, 18, 20 conform to the curvature of the female vagina.

In the exemplary embodiment, the container vessel 10 further includes a pair of inner walls 28 extending inwards from the inner surface of the first side 12 and the second side 14. Each wall 28 curves towards the receiving channel 24. The pair of inner walls 28 couple at an upper region of the container vessel 10. Further, the pair of inner walls 28 are uncoupled at a lower region of the container vessel 10. The pair of inner walls 28 further form an entry way 30 at the opening of the receiving channel 24. The entry way 30 is u-shaped. One skilled in the art would understand the applicant's design is not the exclusive embodiment.

In the exemplary embodiment, the container vessel 10 further includes an outer rim 32. The outer rim 32 is a thinly walled curvature member conforming to the curvature of the vagina. Specifically, the outer rim 32 is positioned on an edging of the first side 12, the second side 14, the third side 16 and the fifth side 20. The outer rim 32 extends beyond and away from the edging of the first side 12, the second side 14, the third side 16 and the fifth side 20. The outer rim 32 further includes an outer surface and an inner surface.

In the exemplary embodiment, the outer rim 32 includes a film 34 composed of a silicon adhesive providing an adhesive layer, positioned over the entirety of the inner surface. The outer rim 32 further includes a protective pad 36 or cover fixable over the film 34. One skilled in the art would understand the applicant's design is not the exclusive embodiment.

In the exemplary embodiment, a collection vessel 50 includes a cylindrical member 52. One skilled in the art would understand another shape or design of the cylindrical member may be provided and the present design is not the exclusive embodiment.

In the exemplary embodiment, the cylindrical member 52 is formed of a material having a high absorptive and/or adsorption rate, and a high permeation rate such that urine can be rapidly wicked and diverted from the receiving channel 24.

The collection vessel 50 further includes a tubing 54. The tubing 54 is positioned and friction fitted within the release passageway 22. One skilled in the art would understand the tubing can be any type of tubing which includes medical quality tubing. The tubing 54 further includes a connector 56. The connector 56 is a normal connector. One skilled in the art would understand other connectors may be attached to the tubing 54.

As assembled, a cylindrical member 52 is positioned within the receiving channel 24 and abuts against an inner surface of the inner walls 28. The tubing 54 is friction fitted within the release passageway 22. A user will remove the protective pad 36 and expose the film 34. The film 34 will then be positioned over an outer surface of the female genitalia, specifically the outer surface of the vagina. Fluid, specifically, urine will flow from the vagina towards the entry way 30 and into the cylindrical member 52. The cylindrical member 52 will absorb the urine, simultaneously, excess urine that is not absorbed by the cylindrical member 52 will flow towards the release passageway 22. The tubing 54 coupled and friction fitted to the release passageway 22 provides suction which moves the unabsorbed liquid toward the release passageway 22 and into the tubing 54.

The above description is only for the specific embodiment of the present invention and is not intended to limit the scope of the present invention. The equivalent changes or modifications made by the structures, features and principles of the present invention should be included in Within the scope of the patent application of the present invention.

What is claimed is:

1. A catheter assembly comprising:
a container vessel with an overall curvature having an outer rim, the container vessel further includes a receiving passageway extending through an upper region of the container vessel;
a collection vessel includes a high absorption member positioned within an inner surface of the container vessel; and the collection vessel further includes a tube fitted within the receiving passageway.

2. The catheter assembly of claim 1, wherein the container vessel includes a first side, a second side, a third side, a fourth side and a fifth side.

3. The catheter assembly of claim 2, wherein the fifth side includes the release passageway extending through the fifth side and into a receiving channel of the container vessel.

4. The catheter assembly of claim 3, wherein the container vessel further includes a pair of inner walls extending inwards from the inner surface of the first side and the second side.

5. The catheter assembly of claim 4, wherein the pair of inner walls couple at the upper region of the container vessel.

6. The catheter assembly of claim 5, wherein the pair of inner walls are uncoupled at a lower region of the container vessel.

7. The catheter assembly of claim 6, wherein the pair of inner walls further includes a u-shaped entry way to the receiving channel.

8. The catheter assembly of claim 7, wherein the outer rim is a thinly walled curvature member.

9. The catheter assembly of claim 8, wherein the outer rim further includes an outer surface and an inner surface.

10. The catheter assembly of claim 1, wherein an inner surface of the outer rim includes a film.

11. The catheter assembly of claim 10, wherein the film is composed of a silicon adhesive covering the entirety of the inner surface.

12. The catheter assembly of claim 11, wherein the outer rim further includes a protective pad fixable over the film.

13. A catheter assembly comprising:
a first side, a second side, a third side, a fourth side and a fifth side forming a container vessel; the container vessel further having a pair of inner walls extending inwards towards an entry way of a receiving channel;
an outer rim positioned around the first side, the second side, the third side and the fifth side; the fifth side includes a receiving passageway extending through to the receiving channel;
an absorbative member coupled within the receiving channel and abutting against an inner surface of the pair of inner walls; and
a tubing positioned within the receiving passageway.

14. The catheter assembly of claim 13, wherein the outer rim includes an outer surface and an inner surface.

15. The catheter assembly of claim 14, wherein the outer rim includes a film.

16. The catheter assembly of claim 15, wherein the film is composed of a silicon adhesive covering the entirety of the inner surface.

17. The catheter assembly of claim 16, wherein the outer rim further includes a protective pad fixable over the film.

18. A catheter assembly comprising:
a container vessel having an outer rim, the outer rim includes a film composed of a silicon adhesive covering the entirety of an inner surface of the outer rim;
the container vessel further includes a receiving passageway extending through an upper region of the container vessel; and
a collection vessel includes a cylindrical member composed of a high absorbative material and positioned within an inner surface of the container vessel.

19. The catheter assembly of claim 18, wherein the outer rim further includes a cover coupled over the film.

20. The catheter assembly of claim 19, wherein a fluid is retained by the cylindrical member while excess fluid drains into the receiving passageway and away from the container vessel.
